(19) United States
(12) Patent Application Publication
(10) Pub. No.: US 2008/0234341 A1
(43) Pub. Date: Sep. 25, 2008

(54) Carisoprodol and Phenytoin Methods

(75) Inventors: Richard H. Roberts, Lakewood, NJ (US); Matthew W. Davis, Erwinna, PA (US)

Correspondence Address: CANTOR COLBURN, LLP, 20 Church Street, 22nd Floor, Hartford, CT, 06103, US

(73) Assignee: MUTUAL PHARMACEUTICAL COMPANY, INC., Philadelphia, PA (US)

(21) Appl. No.: 12/105,646

(22) Filed: Apr. 18, 2008

Related U.S. Application Data
(63) Continuation-in-part of application No. 11/865,143, filed on Oct. 1, 2007.
(60) Provisional application No. 60/827,554, filed on Sep. 29, 2006.

(51) Int. Cl. A61K 31/4164; C07C 271/12

(52) U.S. Cl. 514/385; 560/158

(57) ABSTRACT

A method of using carisoprodol comprises informing a user that co-administration of carisoprodol with steady-state phenyloin results in an increase in free phenyloin blood levels, a decrease in total phenyloin blood levels, or both. In another embodiment, a method of using carisoprodol comprises informing a user that when co-administering carisoprodol with steady-state phenyloin, the level of free phenyloin in a patient serum should be monitored, the level of total phenyloin should be monitored, or both. Also included are methods and articles of manufacture.

### CROSS REFERENCE TO RELATED APPLICATION

This application is a continuation in part of U.S. application Ser. No. 11/865,143 filed Oct. 1, 2007, which claims priority from U.S. Provisional Application Ser. No. 60/827,554 filed Sep. 29, 2006, which is hereby incorporated by reference in its entirety.

### BACKGROUND

Carisoprodol, chemical name N-isopropyl-2-methyl-2-propyl-1,3-propanediol dicarbamate, is a pharmaceutical active agent whose metabolite is meprobamate. Metabolism of carisoprodol by cytochrome P450 is form CYP2C19 has been reported. Carisoprodol was approved by the U.S. FDA on Apr. 9, 1959. It is marketed in the United States under the brand name Soma®, and in the United Kingdom and other countries under the brand name Carisoma®. Carisoprodol is commonly used as a skeletal muscle relaxant.

Carisoprodol is a colorless, crystalline powder, having a mild, characteristic odor and a bitter taste. It is very sparingly soluble in water and freely soluble in alcohol, chloroform, and acetone.

Phenyloin, 5,5-diphenylhydantoin, is an antiepileptic drug useful in the treatment of epilepsy, which is eliminated via metabolism by cytochrome P450 isoforms, including CYP1A2, CYP2C9, CYP2C19, and CYP3A4. Phenyloin may have beneficial effects in the treatment of trigeminal neuralgia in some patients. Phenyloin has a narrow therapeutic index such that too little can lead to insufficient clinical results and excessive phenyloin can lead to phenyloin toxicity. Physicians typically monitor total phenyloin serum levels. The typical clinically effective serum level of total phenyloin is about 10 to about 20 μg/mL. Phenyloin is highly bound to plasma proteins, yet both the efficacy and toxicity of phenyloin are due to the small fraction of unbound phenyloin. The typical clinically effective serum level of unbound or free phenyloin is about 1 to about 2 μg/mL. The recommended initial dose is one 100 mg capsule 3 to 4 times per day, with 300 mg/day dose in three divided doses or one single dose per day.

The present invention addresses the need for improved carisoprodol articles and methods of administering carisoprodol.

### SUMMARY

There is an especially important need for improvements in carisoprodol articles and methods because studies of possible negative or competing interactions with narrow therapeutic index drugs have been limited.

In one embodiment, a method of treating a patient in need of a skeletal muscle relaxant comprises administering to a patient in need thereof a composition comprising carisoprodol, and providing to the patient and/or a medical care worker published material providing information that when co-administering carisoprodol with steady-state phenyloin, the level of free phenyloin in a patient serum sample should be monitored during co-administration.

A method of preventing overdosing a patient with phenyloin comprises co-administering to the patient steady-state phenyloin and a dose of carisoprodol, and informing the patient and/or a medical care worker that co-administration of carisoprodol with steady-state phenyloin can result in an increase in free phenyloin blood levels, total phenyloin blood levels, or both during co-administration.

In another embodiment, a method of administering carisoprodol comprises co-administering to a patient carisoprodol with steady-state phenyloin, and monitoring the free phenyloin concentration in a serum sample from the patient during co-administration.

A method of using carisoprodol or phenyloin comprises informing a user that co-administration of carisoprodol with steady-state phenyloin can result in an increase in free phenyloin blood levels during co-administration.

A method of treating a patient in need of a skeletal muscle relaxant, comprises co-administering to the patient in need thereof carisoprodol and phenyloin, wherein the phenyloin is at steady-state, and monitoring the level of free phenyloin in a patient serum sample during co-administration.

A method of preventing overdosing a patient with phenyloin comprises administering a dose of carisoprodol to a patient having a steady-state blood level of phenyloin, monitoring the level of free phenyloin in a patient serum sample during co-administration with carisoprodol, and optionally adjusting the dose of phenyloin to maintain a safe level of phenyloin, wherein a safe level is about 1 to about 2 ug/mL for free phenyloin and about 10 to about 20 ug/mL for total phenyloin.

In one embodiment, a method of determining a risk of overdosing a patient with phenyloin comprises administering phenyloin to the patient, wherein the phenyloin levels are at steady-state, determining that carisoprodol is administered to the patient, and determining that the patient is at risk for an overdose of phenyloin if it is determined that the patient is administered carisoprodol with phenyloin.

A method of treating a patient in need of a skeletal muscle relaxant comprises co-administering phenyloin with carisoprodol, wherein the phenyloin levels are at steady-state, measuring blood plasma concentrations of phenyloin from the patient 7-10 days after co-administering carisoprodol, determining if free phenyloin, total phenyloin, or both increased, making a record of the level of increase in phenyloin, and reducing or stopping the dose of carisoprodol if the level of phenyloin increases such that the person is at risk of, or is illustrating side effects. In one embodiment, the free and/or total phenyloin concentrations are measured every 7-10 days during co-administration.

In another embodiment, a method of increasing the serum level of free and/or total phenyloin in a patient comprising co-administering phenyloin and carisoprodol, wherein the phenyloin levels are at steady-state, measuring the patient's free and total phenyloin levels 7-10 days after administering carisoprodol, and adjusting the amount of carisoprodol administered or not adjusting the amount of carisoprodol administered so that the patient's free and/or total patient phenyloin level are about 1 to about 2 ug/mL for free phenyloin and about 10 to about 20 ug/mL for total phenyloin.

In another embodiment, a method of preventing overdosing a patient with phenyloin comprises administering carisoprodol daily for a period of at least about 14 days to the patient who is also being administered a dosage strength of phenyloin daily and decreasing by about 85 to 89% the dosage strength of phenyloin being administered to the patient, and optionally measuring the patient's blood plasma levels to determine if the patient's total phenyloin blood level is between about 10 to about 20 ug/mL.

In yet another embodiment, a method of preventing overdosing a patient with phenyloin comprises administering carisoprodol daily for a period of at least about 14 days to the patient that is also being administered about 300 mg of phenyloin daily, decreasing the phenyloin to between about 250 mg and about 270 mg, and optionally measuring the patient's blood plasma levels to determine if the patient's total phenyloin blood level is between about 10 to about 20 ug/mL.

In another embodiment, a method of decreasing the amount of phenyloin necessary to treat a patient in need of phenyloin comprises administering carisoprodol for a period of at least about 14 days and administering about 85% to about 89% of the amount of phenyloin administered to the patient.

These and other embodiments, advantages and features of the present invention become clear when detailed description and examples are provided in subsequent sections.

### DETAILED DESCRIPTION

Carisoprodol is metabolized by cytochrome p450s, including cytochrome p450 isozyme CYP2C19. Other active agents, particularly narrow therapeutic index substances, are also metabolized by cytochrome p450 isozyme CYP2C19. Studies were thus undertaken to determine the effects on plasma concentration, bioavailability, safety, efficacy, or a combination comprising at least one of the foregoing that occur when co-administering carisoprodol and a narrow therapeutic index substance, such as phenyloin. It was unexpectedly discovered by the inventors herein that the blood levels of free phenyloin were significantly increased when phenyloin was co-administered with carisoprodol, both in single-dose and steady-state studies. In addition, the level of total phenyloin was also increased when carisoprodol was dosed to patients taking steady-state phenyloin. In one embodiment, the increase in free phenyloin occurs whether the phenyloin and carisoprodol are co-administered as a single dose. In another embodiment, the increase in free and or total phenyloin occurs when carisoprodol is administered to a patient receiving steady-state phenyloin. Such an increase in free phenyloin blood levels can have a significant effect on the safety and/or efficacy of co-administration of carisoprodol with phenyloin or fosphenyloin. It was also unexpectedly discovered by the inventors herein that the blood levels of total phenyloin were significantly decreased when phenyloin was co-administered with carisoprodol in a single-dose study. Such a decrease in total phenyloin blood levels can have a significant effect on the safety and/or efficacy of co-administration of carisoprodol with phenyloin. Because individuals with epilepsy take phenyloin daily, often for life, it is likely that an individual taking phenyloin will at some point take a skeletal muscle relaxant such as carisoprodol. In addition, people frequently misuse muscle relaxants by taking them for more time than they should (e.g., for longer than two weeks, in the case of carisoprodol), thus increasing the risk of negative effects from co-administration.

An “active agent” means a compound, element, or mixture that when administered to a patient, alone or in combination with another compound, element, or mixture, confers, directly or indirectly, a physiological effect on the patient. The indirect physiological effect may occur via a metabolite or other indirect mechanism. When the active agent is a compound, then salts, solvates (including hydrates), and co-crystals of the free compound or salt, crystalline forms, non-crystalline forms, and any polymorphs of the compound are contemplated herein. Compounds may contain one or more asymmetric elements such as stereogenic centers, stereogenic axes and the like, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In addition, compounds with carbon-carbon double bonds may occur in Z- and E-forms, with all isomeric forms of the compounds. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

All forms (for example solvates, optical isomers, enantiomeric forms, polymorphs, free compound and salts of an active agent) of phenyloin and carisoprodol or other active agent may be employed either alone or in combination.

“Pharmaceutically acceptable salts” includes derivatives of carisoprodol, wherein the carisoprodol is modified by making non-toxic acid or base addition salts thereof, and further refers to pharmaceutically acceptable solvates, including hydrates, of such compounds and such salts. Examples of pharmaceutically acceptable salts include, but are not limited to, mineral or organic acid addition salts of basic residues such as amines; alkali or organic addition salts of acidic residues; and the like, and combinations comprising one or more of the foregoing salts. The pharmaceutically acceptable salts include non-toxic salts and the quaternary ammonium salts of the carisoprodol. For example, non-toxic acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, and combinations comprising one or more of the foregoing salts. Pharmaceutically acceptable organic salts includes salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparginate, glutamate, and the like; and combinations comprising one or more of the foregoing salts.

“Efficacy” means the ability of an active agent administered to a patient to produce a therapeutic effect in the patient.

“Safety” means the incidence or severity of adverse events associated with administration of an active agent, including adverse effects associated with patient-related factors (e.g., age, gender, ethnicity, race, target illness, abnormalities of renal or hepatic function, co-morbid illnesses, genetic characteristics such as metabolic status, or environment) and active agent-related factors (e.g., dose, plasma level, duration of exposure, concomitant medication, or interactions with concomitant medication).

“Enhancing the safety profile” of an active agent means implementing actions or articles designed or intended to help reduce the incidence of adverse events associated with administration of the active agent, including adverse effects associated with patient-related factors (e.g., age, gender, ethnicity, race, target illness, abnormalities of renal or hepatic function, co-morbid illnesses, genetic characteristics such as metabolic status, or environment) and active agent-related factors (e.g., dose, plasma level, duration of exposure, or concomitant medication).

The term “overdose” or overdosing describes the ingestion or application of a drug or other substance in quantities greater than are recommended or generally practiced. An overdose is widely considered harmful and dangerous. In the case of phenyloin, an overdose can have toxic effects such as confusional states referred to as “delirium,” “psychosis,” or “encephalopathy,” or rarely irreversible cerebellar dysfunction. Accordingly, at the first sign of acute toxicity, determination of plasma levels is recommended. Dose reduction of phenyloin therapy is indicated if plasma levels are excessive; if symptoms persist, termination is recommended.

“Active agent interaction” refers to a change in the metabolism of an active agent in a patient that can occur with co-administration of a second active agent.

Administering an active agent with a substance”, “administering an active agent and a substance”, or “co-administering an active agent and a substance” means the active agent and the substance are administered simultaneously in a single dosage form, administered concomitantly in separate dosage forms, or administered in separate dosage forms separated by some amount of time that is within the time in which both the active agent and the substance are within the blood stream of a patient. The active agent and the substance need not be prescribed for a patient by the same medical care worker. Administration of the active agent or the substance can occur via any appropriate route, for example, oral tablets, oral capsules, oral liquids, inhalation, injection, suppositories or topical contact.

“Adverse event” means any untoward medical occurrence in a patient administered an active agent and which does not necessarily have to have a causal relationship with this treatment. An adverse event (AE) can therefore be any unfavorable and unintended sign (including an abnormal laboratory finding, for example), symptom, or disease temporally associated with the use of the active agent, whether or not considered related to the active agent.

“Adverse reaction” means a response to an active agent which is noxious and unintended and which occurs at doses normally used in humans for prophylaxis, diagnosis, or therapy of disease or for modification of physiological function. The unintended response can be an unexpected diminished or enhanced pharmacologic activity or toxicity of the active agent. An adverse reaction also includes any undesirable or unexpected event requiring discontinuation of the active agent, modification of the dose, prolonged hospitalization, or the administration of supportive treatment.

“Affects” include an increase or decrease in degree, level, or intensity; a change in time of onset or duration; a change in type, kind, or effect, or a combination comprising at least one of the foregoing.

“Dosing regimen” means the dose of an active agent taken at a first time by a patient and the interval (time or symptomatic) at which any subsequent doses of the active agent are taken by the patient. The additional doses of the active agent can be different from the dose taken at the first time.

A “dose” means the measured quantity of an active agent to be taken at one time by a patient.

Carisoprodol is a “very sparingly soluble” compound, having a solubility in water of 0.3 to 1.4 mg/mL between 25 to 50° C.

A substance having a “narrow therapeutic index” (NTI) means a substance falling within any definition of narrow therapeutic index as promulgated by the U.S. Food and Drug Administration or any successor agency thereof, for example, a substance having a less than 2-fold difference in median lethal dose (LD50) and median effective dose (ED50) values for the substance, or having a less than 2-fold difference in the minimum toxic concentration and minimum effective concentration in the blood of the substance.

A “dosage form” means a unit of administration of an active agent. Examples of dosage forms include tablets, capsules, injections, suspensions, liquids, emulsions, creams, ointments, suppositories, inhalable forms, transdermal forms, and the like.

The term “effective amount” or “therapeutically effective amount” means an amount effective, when administered to a patient, to provide any therapeutic benefit. A therapeutic benefit may be an amelioration of symptoms, e.g., an amount effective to decrease the symptoms of epilepsy for phenyloin and fosphenyloin. A therapeutic benefit may be an amount suitable to acts as a skeletal muscle relaxant for carisoprodol. The amount that is “effective” will vary from subject to subject, depending on the age and general condition of the individual, the particular active agent, and the like. Thus, it is not always possible to specify an exact “effective amount.” However, an appropriate “effective” amount in any individual case may be determined by one of ordinary skill in the art using routine experimentation. In certain circumstances a patient may not present symptoms of a condition for which the patient is being treated. A therapeutically effective amount of an active agent may also be an amount sufficient to provide a significant positive effect on any indicium of a disease, disorder, or condition. A significant effect on an indicium of a disease, disorder, or condition is statistically significant in a standard parametric test of statistical significance, for example Student's T-test, where p<0.05.

By “oral dosage form” is meant to include a unit dosage form for oral administration. An oral dosage form may optionally comprise a plurality of subunits such as, for example, microcapsules or microtablets. Multiple subunits may be packaged for administration in a single dose. By “subunit” is meant to include a composition, mixture, particle, pellet, etc., that can provide an oral dosage form alone or when combined with other subunits.

A dissolution profile is a plot of the cumulative amount of active agent released as a function of time. A dissolution profile can be measured utilizing the Drug Release Test <724>, which incorporates standard test USP 26 (Test <711>). A profile is characterized by the test conditions selected such as, for example, apparatus type, shaft speed, temperature, volume, and pH of the dissolution medium. More than one dissolution profile may be measured. For example, a first dissolution profile can be measured at a pH level approximating that of the stomach, and a second dissolution profile can be measured at a pH level approximating that of one point in the intestine or several pH levels approximating multiple points in the intestine.

A highly acidic pH may be employed to simulate the stomach and a less acidic to basic pH may be employed to simulate the intestine. By the term “highly acidic pH” is meant a pH of about 1 to about 4. A pH of about 1.2, for example, can be used to simulate the pH of the stomach. By the term “less acidic to basic pH” is meant a pH of greater than about 4 to about 7.5, specifically about 6 to about 7.5. A pH of about 6 to about 7.5, specifically about 6.8, can be used to simulate the pH of the intestine.

By “immediate-release” is meant a conventional or non-modified release in which greater than or equal to about 75% of the active agent is released within two hours of administration, specifically within one hour of administration.

By “controlled-release” is meant a dosage form in which the release of the active agent is controlled or modified over a period of time. Controlled can mean, for example, sustained-, delayed- or pulsed-release at a particular time. Alternatively, controlled can mean that the release of the active agent is extended for longer than it would be in an immediate-release dosage form, e.g., at least over several hours.

Dosage forms can be combination dosage forms having both immediate-release and controlled-release characteristics, for example, a combination of immediate-release pellets and controlled-release pellets. The immediate-release portion of a combination dosage form may be referred to as a loading dose.

“Bioavailability” means the extent or rate at which an active agent is absorbed into a living system or is made available at the site of physiological activity. For active agents that are intended to be absorbed into the bloodstream, bioavailability data for a given formulation may provide an estimate of the relative fraction of the administered dose that is absorbed into the systemic circulation. “Bioavailability” can be characterized by one or more pharmacokinetic parameters.

“Pharmacokinetic parameters” describe the in vivo characteristics of an active agent (or surrogate marker for the active agent) over time, such as plasma concentration (C), Cₘₐₓ, Cₙ, C₂₄, Tₘₐₓ, and AUC. “Cₘₐₓ” is the measured concentration of the active agent in the plasma at the point of maximum concentration. “Cₙ” is the measured concentration of an active agent in the plasma at about n hours after administration. “C₂₄” is the measured concentration of an active agent in the plasma at about 24 hours after administration. The term “Tₘₐₓ” refers to the time at which the measured concentration of an active agent in the plasma is the highest after administration of the active agent. “AUC” is the area under the curve of a graph of the measured concentration of an active agent (typically plasma concentration) vs. time, measured from one time point to another time point. For example AUC₀₋ₜ is the area under the curve of plasma concentration versus time from time 0 to time t. The AUC_{0-∞} or AUC_{0-INF} is the calculated area under the curve of plasma concentration versus time from time 0 to time infinity.

Under U.S. FDA guidelines, two products or methods (e.g., dosing under non-fasted versus fasted conditions) are bioequivalent if the 90% Confidence Intervals (CI) for a log transformed geometric mean of AUC_{0-FNF}, and Cₘₐₓ are 0.80 to 1.25 (Tₘₐₓ measurements are not relevant to bioequivalence for regulatory purposes). To show bioequivalency between two compounds or administration conditions pursuant to Europe's EMEA guidelines, the 90% CI for a log transformed geometric mean of AUC_{0-INF}, must be 0.80 to 1.25 and the 90% CI for a log transformed geometric mean of Cₘₐₓ must be 0.70 to 1.43.

As used herein, pharmacokinetic parameters can be measured for single-dose administration or steady-state administration of one or both active agents. Unless specified as steady-state parameters, all pharmacokinetic parameters disclosed herein are measured after single-dose administration. In single-dose administration, both active agents (e.g., phenyloin and carisoprodol) are co-administered as a single dose. In contrast to single-dose administration, steady-state administration is the condition under which one agent (e.g., phenyloin or carisoprodol) is administered for a period of time to establish a substantially constant blood level of the first active agent prior to introducing single-doses of the second active agent (e.g., carisoprodol or phenyloin). In one embodiment, steady-state administration comprises administration for at least 5 half lives after starting dosing, or, in the case of phenyloin, approximately 7-10 days of administration.

Information as disclosed herein may include information that two administration methods are bioequivalent under FDA guidelines, are substantially bioequivalent, or have insignificant differences in their pharmacokinetic parameters.

Certain formulations described herein may be “coated”. The coating may be a suitable coating, such as, a functional or a non-functional coating, or multiple functional and/or non-functional coatings. By “functional coating” is meant to include a coating that modifies the release properties of the total formulation, for example, a sustained-release coating. By “non-functional coating” is meant to include a coating that is not a functional coating, for example, a cosmetic coating. A non-functional coating can have some impact on the release of the active agent due to the initial dissolution, hydration, perforation of the coating, etc., but would not be considered to be a significant deviation from the non-coated composition.

“Informing” means referring to or providing, published material, for example, providing an active agent with published material to a user; or presenting information orally, for example, by presentation at a seminar, conference, or other educational presentation, by conversation between a pharmaceutical sales representative and a medical care worker, or by conversation between a medical care worker and a patient; or demonstrating the intended information to a user for the purpose of comprehension.

“Labeling” means all labels or other means of written, printed, graphic, electronic, verbal, or demonstrative communication that is upon a pharmaceutical product or a dosage form or accompanying such pharmaceutical product or dosage form.

A “medical care worker” means a worker in the health care field who may need or utilize information regarding an active agent including a dosage form thereof, including information on safety, efficacy, dosing, administration, or pharmacokinetics. Examples of medical workers include physicians, pharmacists, physician's assistants, nurses, aides, caretakers (which can include family members or guardians), emergency medical workers, and veterinarians.

A “patient” means a human or non-human animal in need of medical treatment. Medical treatment can include treatment of an existing condition, such as a disease or disorder, prophylactic or preventative treatment, or diagnostic treatment. In some embodiments the patient is a human patient.

A “pharmaceutical supplier” means a person (other than a medical care worker), business, charitable organization, governmental organization, or other entity involved in the transfer of active agent, including a dosage form thereof, between entities, for profit or not. Examples of pharmaceutical suppliers include pharmaceutical distributors, pharmacy chains, pharmacies (online or physical), hospitals, HMOs, supermarkets, the Veterans Administration, or foreign businesses or individuals importing active agent into the United States.

A “user” means a patient, a medical care worker, or a pharmaceutical supplier.

A “product” or “pharmaceutical product” means a dosage form of an active agent plus published material and optionally packaging.

“Providing” means giving, administering, selling, distributing, transferring (for profit or not), manufacturing, compounding, or dispensing.

“Published material” means a medium providing information, including printed, audio, visual, or electronic medium, for example a flyer, an advertisement, a product insert, printed labeling, an internet web site, an internet web page, an internet pop-up window, a radio or television broadcast, a compact disk, a DVD, an audio recording, or other recording or electronic medium.

“Product insert” means the professional labeling (prescribing information) for a pharmaceutical product, a patient package insert for the pharmaceutical product, or a medication guide for the pharmaceutical product.

“Professional labeling” or “prescribing information” means the official description of a pharmaceutical product approved by a regulatory agency (e.g., FDA or EMEA) regulating marketing of the pharmaceutical product, which includes a summary of the essential scientific information needed for the safe and effective use of the drug, such as, for example indication and usage; dosage and administration; who should take it; adverse events (side effects); instructions for use in special populations (pregnant women, children, geriatric, etc.); safety information for the patient, and the like.

“Patient package insert” means information for patients on how to safely use a pharmaceutical product that is part of the FDA-approved labeling. It is an extension of the professional labeling for a pharmaceutical product that may be distributed to a patient when the product is dispensed which provides consumer-oriented information about the product in lay language, for example it may describe benefits, risks, how to recognize risks, dosage, or administration.

“Medication Guide” means an FDA-approved patient labeling for a pharmaceutical product conforming to the specifications set forth in 21 CFR 208 and other applicable regulations which contains information for patients on how to safely use a pharmaceutical product. A medication guide is scientifically accurate and is based on, and does not conflict with, the approved professional labeling for the pharmaceutical product under 21 CFR 201.57, but the language need not be identical to the sections of approved labeling to which it corresponds. A medication guide is typically available for a pharmaceutical product with special risk management information.

Food typically means a solid food or mixed solid/liquid food with sufficient bulk and fat content that it is not rapidly dissolved and absorbed in the stomach. In one embodiment, food means a meal, such as breakfast, lunch or dinner. The terms “taken with food”, “fed” and “non-fasted” are equivalent and are as given by FDA guidelines and criteria. In one embodiment, with food means that the dosage form is administered to a patient between about 30 minutes prior to about 2 hours after eating a meal. In another embodiment, with food means that the dosage form is administered at substantially the same time as the eating the meal.

The terms “without food”, “fasted” and “an empty stomach” are equivalent and are as given by FDA guidelines and criteria. In one embodiment, fasted means the condition wherein no food is consumed within 1 hour prior to administration of the dosage form or 2 hours after administration of the dosage form. In another embodiment, fasted means the condition wherein no food is consumed within 1 hour prior to administration of the dosage form to 2 hours after administration of the dosage form.

Carisoprodol is metabolized by cytochrome p450s, including cytochrome p450 isozyme CYP2C19. Other active agents, particularly narrow therapeutic index substances, are also metabolized by cytochrome p450 isozyme CYP2C19. Studies were thus undertaken to determine the effects on plasma concentration, bioavailability, safety, efficacy, or a combination comprising at least one of the foregoing that occur when co-administering carisoprodol and a narrow therapeutic index substance, specifically phenyloin.

Phenyloin is eliminated via metabolism by cytochrome P450 isoforms, including CYP1A2, CYP2C9, CYP2C19, and CYP3A4. Phenyloin is highly bound (e.g., about 90% bound) to plasma proteins in the bloodstream such as albumin. For phenyloin, drug response is dependent on free drug concentration, that is, the about 10% of the drug that is not bound to plasma proteins. Without being held to theory, it is believed that the average person has 10 to 20% free phenyloin. If the level increases to over 20%, there is an increased risk of adverse events. Since it is the free phenyloin in plasma which equilibrates with the site of pharmacological or toxic response, a slight change in the extent of binding leads to a substantial percentage change in free phenyloin concentration, which in turn causes a very significant alteration in response. Slight changes in the binding of highly bound drugs to plasma proteins leads to significant changes in the clinical response, and in some cases causes a toxic response. In addition, the amount of free protein will vary from person to person, so that checking blood levels after you potentially perturb the levels of free protein is critical.

Due to the narrow therapeutic index of phenyloin, plus wide individual variability in the rate of phenyloin metabolism and clearance, therapeutic drug monitoring during treatment with phenyloin is typically recommended. Insufficient blood levels may lead to inadequate symptom management, while excess blood levels can lead to dangerous toxicity. However, because the measurement of total phenyloin blood levels is easier than the measurement of free phenyloin blood levels, physicians typically monitor total phenyloin blood levels.

In one embodiment, monitoring the patient comprises monitoring the patient's plasma concentration of phenyloin, specifically free and/or total phenyloin; monitoring the patient for symptoms of an active agent interaction between carisoprodol and phenyloin; monitoring the patient for an adverse reaction (e.g., toxicity) resulting from administration of carisoprodol and phenyloin; monitoring the patient for an adverse reaction (e.g., toxicity) resulting from administration of phenyloin; or monitoring the patient for decreased efficacy of phenyloin. In one embodiment, the phenyloin concentration is at steady-state prior to administration of carisoprodol. Monitoring is performed, for example, daily in the early stages of treatment, to once yearly once a treatment protocol is established. In one embodiment, monitoring is performed once every 7 to 14 days.

In another embodiment, the method comprises determining free and/or total phenyloin blood levels, making a record of the level of increase in phenyloin, and reducing or stopping the dose of carisoprodol if the level of phenyloin increases such that the person is at risk of, or is illustrating side effects. In one embodiment, the record is a patient file such as a chart or computer file. In one embodiment, the phenyloin is at steady-state.

In one embodiment, a method of treating a patient further comprises adjusting the dosage strength of phenyloin in response to the monitoring, adjusting the dosage frequency of phenyloin in response to the monitoring, observing the patient for signs of toxicity, or a combination thereof. In one embodiment, the phenyloin is at steady-state.

Total phenyloin in a serum sample can be measured, for example, in a turbidimetry assay. Turbidimetry measures the diminution in power of a collimated light beam as a result of scattering particles in solution, measured for example in a spectrophotometer. The principle of the method is the competition of phenyloin in a serum sample with phenyloin labeled latex particles for anti-phenyloin antibodies. Phenyloin bound to latex reacts with anti-phenyloin antibodies to form immune complexes in an appropriate buffer. Phenyloin in a serum sample competes with phenyloin-latex and this competitive reaction can be measured by turbidimetry. The absorbance is inversely related to the phenyloin concentration in the serum sample. The actual concentration is determined from a calibration curve prepared with standards of known concentration.

Free phenyloin in a serum sample can be measured, for example, in a fluorescence polarization immunoassay (FPIA). First, protein-bound phenyloin is separated from free phenyloin using, for example, ultrafiltration. Then the isolated free phenyloin is measured by FPIA. FPIA takes advantage of the increased polarization non-random propagation of emission of fluorescent light emissions when a fluorescently labeled antigen is bound by reagent antibody. The higher the concentration of unlabeled patient phenyloin in the test mixture, the less bound fluorescent phenyloin is present and, consequently, the lower the polarization of the fluorescent light emission. Standard calibration yields quantitative results.

It has been unexpectedly discovered that co-administration of carisoprodol with phenyloin in a single-dose or steady-state study leads to a statistically significant increase in free phenyloin blood levels. Concurrently, there is a decrease in total phenyloin blood levels in a single-dose co-administration study. In a steady-state phenyloin study, both the free and total steady-state levels of phenyloin are increased when carisoprodol is co-administered with the steady-state phenyloin. Such a change is clinically significant as small changes in free phenyloin blood levels can lead to significant changes in clinical response and in some cases causes a toxic response. Also, the lower level of total phenyloin may cause a physician to erroneously infer that free phenyloin has also decreased.

In one embodiment, a method of using carisoprodol comprises informing a user that co-administration of carisoprodol with steady-state phenyloin results in an increase in free and/or total phenyloin blood levels during co-administration. The co-administration can lead to changes in the safety and/or efficacy of the carisoprodol, phenyloin, and/or both. The carisoprodol can be a single dose of carisoprodol or more than one dose of carisoprodol administered over a period of one or more days.

In another embodiment, a method of using carisoprodol comprises informing a user that single-dose co-administration of carisoprodol with phenyloin results in an increase in free phenyloin blood levels. In another embodiment, single-dose co-administration of carisoprodol with phenyloin results in a decrease in total phenyloin blood levels. The co-administration can lead to changes in the safety and/or efficacy of the carisoprodol, phenyloin, or both.

Changes in safety and/or efficacy include changes in clinical response and/or a toxic response. When co-administering carisoprodol with phenyloin, the level of free phenyloin in a patient serum should be monitored, the level of total phenyloin in a patient serum should be monitored, or both. In one embodiment, the level of free phenyloin is measured. In another embodiment, a method of using carisoprodol comprises informing a user that when co-administering carisoprodol with phenyloin, the level of free phenyloin in a patient serum should be monitored, the level of total phenyloin in a patient serum should be monitored, or both.

Based on the data presented herein, it is recommended that the free and/or total, specifically the free blood levels, of phenyloin be monitored regularly when phenyloin is co-administered with carisoprodol. In one embodiment, the blood levels of phenyloin, specifically steady-state phenyloin, remain in an acceptable range when phenyloin is co-administered with carisoprodol, thus no change in dosage and administration is required. In another embodiment, the blood levels of phenyloin increase when phenyloin, specifically steady-state phenyloin, is co-administered with carisoprodol. In this embodiment, the dose of carisoprodol or phenyloin is optionally decreased or even ceased in the case of carisoprodol.

Without being held to theory, phenyloin exhibits Michaelis-Menten (nonlinear) kinetics, so the lower the level of phenyloin a person is on, an increase in phenyloin would have a greater effect than an increase at higher levels (because the kinetics curve is “steeper” at lower levels). In one embodiment, when co-administering phenyloin and carisoprodol, it is recommended to monitor patients for phenyloin “toxicity” effects when carisoprodol is given and/or that phenyloin serum levels should also be measured to indicate whether or not a patient has exceeded the typical clinically effective serum level of total phenyloin of about 10 to about 20 μg/m and the typical clinically effective serum level of unbound or free phenyloin of about 1 to about 2 μg/mL, or any other level or levels that are deemed to not cause “toxicity” effects in the patient being measured.

In one embodiment, a method of using carisoprodol, comprises obtaining carisoprodol from a container providing information that when co-administering carisoprodol with phenyloin, the level of free phenyloin in a patient serum should be monitored, the level of total phenyloin in a patient serum should be monitored, or both. Specifically, the level of free phenyloin in a patient serum should be monitored. In another embodiment, a method of manufacturing a carisoprodol pharmaceutical composition comprises packaging a carisoprodol pharmaceutical formulation along with information that when co-administering carisoprodol with phenyloin, the level of free phenyloin in a patient serum should be monitored, the level of total phenyloin in a patient serum should be monitored, or both. In yet another embodiment, an article of manufacture comprises a container holding a dosage form of carisoprodol, wherein the container is associated with printed labeling instructions advising that when co-administering carisoprodol with phenyloin, the level of free phenyloin in a patient serum should be monitored, the level of total phenyloin in a patient serum should be monitored, or both.

In one embodiment, a method of using phenyloin comprises informing a user that co-administration of carisoprodol with steady-state phenyloin can change the safety and/or efficacy of phenyloin. Changes in safety and/or efficacy include changes in clinical response and/or a toxic response. In another embodiment, a method of using phenyloin comprises informing a user that single-dose co-administration of carisoprodol with steady-state phenyloin results in an increase in free phenyloin blood levels, a decrease in total phenyloin blood levels, or both, which can change the safety and/or efficacy of phenyloin. In yet another embodiment, a method of using phenyloin comprises informing a user that co-administration of carisoprodol with steady-state phenyloin results in an increase in free and/or total phenyloin blood levels, which can change the safety and/or efficacy of phenyloin during co-administration. Because it is the free phenyloin that causes both the therapeutic and toxic effects of phenyloin, when co-administering phenyloin with carisoprodol, the level of free phenyloin in a patient serum should be monitored, the level of total phenyloin in a patient serum should be monitored, or both. Thus, in another embodiment, a method of using phenyloin comprises informing a user that when co-administering carisoprodol with phenyloin, the level of free phenyloin in a patient serum should be monitored, the level of total phenyloin in a patient serum should be monitored, or both.

In one embodiment, a method of using phenyloin comprises obtaining phenyloin from a container providing information that when co-administering carisoprodol with steady-state phenyloin, the level of free phenyloin in a patient serum should be monitored, the level of total phenyloin in a patient serum should be monitored, or both. In another embodiment, a method of manufacturing a phenyloin pharmaceutical composition comprises packaging a phenyloin pharmaceutical formulation along with information that when co-administering carisoprodol with steady-state phenyloin, the level of free phenyloin in a patient serum should be monitored, the level of total phenyloin in a patient serum should be monitored, or both. In yet another embodiment, an article of manufacture comprises a container holding a dosage form of phenyloin, wherein the container is associated with printed labeling instructions advising that when co-administering carisoprodol with steady-state phenyloin, the level of free phenyloin in a patient serum should be monitored the level of total phenyloin in a patient serum should be monitored, or both.

In one embodiment, informing comprises providing information that co-administration of carisoprodol for a period of 14 days with steady-state phenyloin resulted in a 14% increase in the Cₘₐₓ for free phenyloin and an 18% increase in the AUC₀₋ₜ for free phenyloin during co-administration. In another embodiment, informing comprises providing information that co-administration of carisoprodol for a period of 14 days with steady-state phenyloin resulted in a 12% increase in the Cₘₐₓ for total phenyloin and an 18% increase in the AUC₀₋ₜ for total phenyloin during co-administration.

In another embodiment, informing comprises providing information that co-administration of carisoprodol for a period of 14 days with steady-state phenyloin resulted in a 14% increase in the Cₘₐₓ for free phenyloin and an 18% increase in the AUC₀₋ₜ for free phenyloin.

In one embodiment, the published material informs that, in a single-dose co-administration study, the geometric mean of Cₘₐₓ for free phenyloin when phenyloin is administered alone is not bioequivalent to that when phenyloin is co-administered with carisoprodol. The published material optionally further informs that the geometric mean of Cₘₐₓ for total phenyloin when phenyloin is administered alone is bioequivalent to that when phenyloin is co-administered with carisoprodol.

In another embodiment, the published material informs that in a single-dose co-administration study, co-administration of phenyloin with carisoprodol results in an increase in Cₘₐₓ for free phenyloin. The published material optionally further informs that co-administration of phenyloin with carisoprodol results in a decrease in Cₘₐₓ for total phenyloin.

In some embodiments, the published material informs that in a co-administration study of carisoprodol with steady-state phenyloin including 21 patients, the geometric mean of Cₘₐₓ for total phenyloin when phenyloin was administered alone was 13604.17 ng/ml, and the geometric mean of Cₘₐₓ for total phenyloin when phenyloin was co-administered with carisoprodol was 15556.59 ng/ml. In other embodiments, the published material informs that in a co-administration study of carisoprodol with steady-state phenyloin including 21 patients, the geometric mean of Cₘₐₓ for free phenyloin when phenyloin was administered alone was 929.76 ng/ml, and the geometric mean of Cₘₐₓ for free phenyloin when phenyloin was co-administered with carisoprodol was 1037.37 ng/ml. In one embodiment, the printed material informs that during co-administration of carisoprodol with steady-state phenyloin, the lower limit of the 90% confidence interval of the ratio of Cₘₐₓ for free phenyloin when phenyloin was administered alone to free phenyloin when phenyloin was co-administered with carisoprodol was 85.46%. In another embodiment, the printed material informs that during co-administration of carisoprodol with steady-state phenyloin the lower limit of the 90% confidence interval of the ratio of Cₘₐₓ for total phenyloin when phenyloin was administered alone to free phenyloin when phenyloin was co-administered with carisoprodol was 93.3%.

In other embodiments, the published material informs that in a single-dose co-administration study of 12 patients, the geometric mean of Cₘₐₓ for total phenyloin when phenyloin was administered alone was 3222.2 ng/ml, and the geometric mean of Cₘₐₓ for total phenyloin when phenyloin was co-administered with carisoprodol was 2801.38 ng/ml. In other embodiments, the published material informs that that in a in a single-dose co-administration study of 12 patients, the geometric mean of Cₘₐₓ for free phenyloin when phenyloin was administered alone was 282.76 ng/ml, and the geometric mean of Cₘₐₓ for free phenyloin when phenyloin was co-administered with carisoprodol was 322.25 ng/ml. In one embodiment, the printed material informs that in a single-dose co-administration study, the lower limit of the 90% confidence interval of the ratio of Cₘₐₓ for free phenyloin when phenyloin was administered alone to free phenyloin when phenyloin was co-administered with carisoprodol was 69.5%.

In one embodiment, the published material informs that in a single-dose co-administration study, the geometric mean of AUC₀₋ₜ for free phenyloin when phenyloin is administered alone is not bioequivalent to that when phenyloin is co-administered with carisoprodol. The published material optionally further informs that in a single-dose co-administration study, the geometric mean of AUC₀₋ₜ for total phenyloin when phenyloin is administered alone is bioequivalent to that when phenyloin is co-administered with carisoprodol.

In another embodiment, the published material informs that co-administration of phenyloin with carisoprodol results in an increase in AUC₀₋ₜ for free phenyloin. The published material optionally further informs that co-administration of phenyloin with carisoprodol in a single-dose co-administration study results in a decrease in AUC₀₋ₜ for total phenyloin.

In one embodiment, the published material informs that the geometric mean of AUC₀₋ₜ for total and/or free phenyloin when phenyloin is administered alone is bioequivalent to that when phenyloin is co-administered with carisoprodol. In some embodiments, the published material informs that in a study of 12 patients, the geometric mean of AUC₀₋ₜ for total phenyloin when phenyloin was administered alone was 97342.7 hr*ng/ml, and the geometric mean of AUC₀₋ₜ for total phenyloin when phenyloin was co-administered with carisoprodol was 91468.8 hr*ng/ml. In some embodiments, the published material informs that in a study of 12 patients, the geometric mean of AUC₀₋ₜ for free phenyloin when phenyloin was administered alone was 5150.25 hr*ng/ml, and the geometric mean of AUC₀₋ₜ for free phenyloin when phenyloin was co-administered with carisoprodol was 5559.62 hr*ng/ml, In one embodiment, the printed material informs that the lower limit of the 90% confidence interval of the ratio of AUC₀₋ₜ for free phenyloin when phenyloin is administered alone to free phenyloin when phenyloin is co-administered with carisoprodol is 75.34%.

In one embodiment, the published material informs that the geometric mean of AUC_{0-INF} for total and/or free phenyloin when phenyloin was administered alone is bioequivalent to that when phenyloin was co-administered with carisoprodol. In some embodiments, the published material informs that in a study of 12 patients, the geometric mean of AUC_{0-INF} for total phenyloin when phenyloin was administered alone was 133987.2 hr*ng/ml, and the geometric mean of AUC_{0-INF} for total phenyloin when phenyloin was co-administered with carisoprodol was 132006.5 hr*ng/ml. In some embodiments, the published material informs that that in a study of 12 patients, the geometric mean of AUC_{0-INF} for free phenyloin when phenyloin was administered alone was 12927.88 hr*ng/ml, and the geometric mean of AUC_{0-INF} for free phenyloin when phenyloin was co-administered with carisoprodol was 100673.26 hr*ng/ml.

In some embodiments, the published material informs that in a study of 21 patients administered carisoprodol in the presence of steady-state phenyloin, the geometric mean of AUC₀₋ₜ for total phenyloin when phenyloin was administered alone was 242582.26 hr*ng/ml, and the geometric mean of AUC₀₋ₜ for total phenyloin when phenyloin was co-administered with carisoprodol was 285541.72 hr*ng/ml. In some embodiments, the published material informs that that in a study of 21 patients administered carisoprodol in the presence of steady-state phenyloin, the geometric mean of AUC₀₋ₜ for free phenyloin when phenyloin was administered alone was 14733.48 hr*ng/ml, and the geometric mean of AUC₀₋ₜ for free phenyloin when phenyloin was co-administered with carisoprodol was 17416.69 hr*ng/ml. In one embodiment, the printed material informs that that in a study of 21 patients administered carisoprodol in the presence of steady-state phenyloin, the lower limit of the 90% confidence interval of the ratio of AUC₀₋ₜ for free phenyloin when phenyloin is administered alone to free phenyloin when phenyloin is co-administered with carisoprodol is 90.88%.

In some embodiments, the published material informs that in a study of 21 patients administered carisoprodol in the presence of steady-state phenyloin, the geometric mean of AUC_{0-INF} for total phenyloin when phenyloin was administered alone was 662587.00 hr*ng/ml, and the geometric mean of AUC_{0-INF} for total phenyloin when phenyloin was co-administered with carisoprodol was 843759.68 hr*ng/ml. In some embodiments, the published material informs that that in a study of 21 patients administered carisoprodol in the presence of steady-state phenyloin, the geometric mean of AUC_{0-INF} for free phenyloin when phenyloin was administered alone was 32701.49 hr*ng/ml, and the geometric mean of AUC_{0-INF} for free phenyloin when phenyloin was co-administered with carisoprodol was 45571.41 hr*ng/ml.

In the foregoing embodiments, the methods optionally further comprise administering providing the user carisoprodol, phenyloin and/or fosphenyloin.

The informing is, for example, by reference to published material; by reference to a package active agent insert, a flyer or an advertisement; by presentation of information at a seminar, conference, or other educational presentation; or by a conversation between a pharmaceutical sales representative and the medical care worker. Informing comprises, for example, providing published material comprising a discussion that when co-administering carisoprodol and phenyloin, the level of free phenyloin in a patient serum should be monitored.

A method of preventing overdosing a patient with phenyloin comprises administering a dose of carisoprodol to a patient having a steady-state blood level of phenyloin, monitoring the level of free phenyloin in a patient serum sample during co-administration with carisoprodol, and optionally adjusting the dose of phenyloin to maintain a safe level of phenyloin, wherein a safe level is about 1 to about 2 ug/mL for free phenyloin and about 10 to about 20 ug/mL for total phenyloin.

In another embodiment, a method of determining a risk of overdosing a patient with phenyloin comprises administering phenyloin to the patient, wherein the phenyloin level is at steady-state, determining that carisoprodol is administered to the patient, and determining that the patient is at risk for an overdose of phenyloin if it is determined that the patient is administered carisoprodol with phenyloin.

In yet another embodiment, a method of treating a patient in need of a skeletal muscle relaxant comprises co-administering phenyloin with carisoprodol, wherein the phenyloin level is at steady-state, measuring blood plasma concentrations of phenyloin from the patient 7-14 days after co-administering carisoprodol, determining if free phenyloin, total phenyloin, or both increased, making a record of the level of increase in phenyloin, and reducing or stopping the dose of carisoprodol if the level of phenyloin increases such that the person is at risk of, or is illustrating side effects. Exemplary side effects include delirium, psychosis, encephalopathy, or irreversible cerebellar dysfunction.

In another embodiment, a method of increasing the serum level of free and/or total phenyloin in a patient comprising co-administering steady-state phenyloin and carisoprodol, measuring the patient's free and total phenyloin levels 7-10 days after administering carisoprodol, and adjusting the amount of carisoprodol administered or not adjusting the amount of carisoprodol administered so that the patient's free and/or total patient phenyloin level are about 1 to about 2 ug/mL for free phenyloin and about 10 to about 20 ug/mL for total phenyloin. In another embodiment, a method of increasing the serum level of free and/or total phenyloin in a patient comprises co-administering steady-state phenyloin and carisoprodol, measuring the patient's free and total phenyloin levels 7-10 days after administering carisoprodol, and adjusting the amount of phenyloin administered or not adjusting the amount of phenyloin administered so that the patient's free and/or total patient phenyloin level are about 1 to about 2 ug/mL for free phenyloin and about 10 to about 20 ug/mL for total phenyloin.

A method of reevaluating dosing when administering carisoprodol to a patient in need of a skeletal muscle relaxant comprises co-administering carisoprodol and phenyloin to the patient, wherein the phenyloin level is at steady-state; determining whether the patient's free phenyloin level and total phenyloin level are within a safe range, wherein a safe range is about 1 to about 2 ug/mL for free phenyloin and about 10 to about 20 ug/mL for total phenyloin; and increasing or decreasing the dose or frequency of carisoprodol administered to the patient if the free phenyloin levels, total phenyloin levels, or both, are outside the safe range.

In another embodiment, a method of increasing the serum level of free and/or total phenyloin in a patient comprises co-administering phenyloin and carisoprodol, wherein the phenyloin levels are at steady-state, measuring the patient's free and total phenyloin levels 7-10 days after administering carisoprodol, and adjusting the amount of phenyloin administered or not adjusting the amount of phenyloin administered so that the patient's free and/or total patient phenyloin level are about 1 to about 2 ug/mL for free phenyloin and about 10 to about 20 ug/mL for total phenyloin.

In another embodiment, a method of using phenyloin, comprises co-administering phenyloin and carisoprodol, wherein the phenyloin is at steady-state, measuring the patient's free and total phenyloin levels 7-10 days after administering carisoprodol, and decreasing the amount of phenyloin administered so that the patient's free and/or total patient phenyloin level are about 1 to about 2 ug/mL for free phenyloin and about 10 to about 20 ug/mL for total phenyloin.

In one embodiment a method of using phenyloin comprises, co-administering steady-state phenyloin and steady-state carisoprodol, measuring the patient's free and total phenyloin levels 7-10 days after co-administration, and stopping carisoprodol administration when the patient's free and/or total patient phenyloin level is outside of about 1 to about 2 ug/mL for free phenyloin and about 10 to about 20 ug/mL for total phenyloin. Stopping carisoprodol administration means discontinuing dosing with carisoprodol for the duration of phenyloin administration. In another embodiment, a method of using phenyloin comprises co-administering steady-state phenyloin and steady-state carisoprodol, and measuring the patient's free and/or total phenyloin level if carisoprodol administration is discontinued.

In another embodiment, a method of preventing overdosing a patient with phenyloin comprises administering carisoprodol daily for a period of at least about 14 days to the patient who is also being administered a dosage strength of phenyloin daily and decreasing by about 85 to 89% the dosage strength of phenyloin being administered to the patient, and optionally measuring the patient's blood plasma levels to determine if the patient's total phenyloin blood level is between about 10 to about 20 ug/mL.

In yet another embodiment, a method of preventing overdosing a patient with phenyloin comprises administering carisoprodol daily for a period of at least about 14 days to the patient that is also being administered about 300 mg of phenyloin daily, decreasing the phenyloin to between about 250 mg and about 270 mg, and optionally measuring the patient's blood plasma levels to determine if the patient's total phenyloin blood level is between about 10 to about 20 ug/mL.

In another embodiment, a method of decreasing the amount of phenyloin necessary to treat a patient in need of phenyloin comprises administering carisoprodol for a period of at least about 14 days and administering about 85% to about 89% of the amount of phenyloin administered to the patient.

Solid dosage forms for oral administration include, but are not limited to, capsules, tablets, pills, powders, and granules. In such solid dosage forms, the active agent may be admixed with one or more of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, and combinations comprising one or more of the foregoing additives. For capsules, tablets, and pills, the dosage forms may also comprise buffering agents.

The dosage forms described herein may be coated with a functional or non-functional coating. The coating may comprise about 0 wt % to about 40 wt % of the composition. The coating material may include a polymer, such as a film-forming polymer including, for example, methyl cellulose, ethyl cellulose, hydroxypropyl cellulose, hydroxypropyl methyl cellulose, hydroxybutyl methyl cellulose, cellulose acetate, cellulose propionate, cellulose acetate propionate, cellulose acetate butyrate, cellulose acetate phthalate, carboxymethyl cellulose, cellulose triacetate, cellulose sulphate sodium salt, poly(methyl methacrylate), poly(ethyl methacrylate), poly (butyl methacrylate), poly(isobutyl methacrylate), poly(hexyl methacrylate), poly (phenyl methacrylate), poly(methyl acrylate), poly(isopropyl acrylate), poly (isobutyl acrylate), poly(octadecyl acrylate), poly(ethylene), poly(ethylene) low density, poly(ethylene)high density, (poly propylene), poly(ethylene glycol poly (ethylene oxide), poly(ethylene terephthalate), poly(vinyl alcohol), poly(vinyl isobutyl ether), poly(viny acetate), poly(vinyl chloride), polyvinyl pyrrolidone, and combinations comprising one or more of the foregoing polymers.

In some applications, the polymer can be a water-insoluble polymer. Water insoluble polymers include ethyl cellulose or dispersions of ethyl cellulose, acrylic and/or methacrylic ester polymers, cellulose acetates, butyrates or propionates or copolymers of acrylates or methacrylates having, for example, a low quaternary ammonium content, and the like, and combinations comprising one or more of the foregoing polymers.

In controlled-release applications, for example, the coating can be a hydrophobic polymer that modifies the release properties of the API from the formulation. Suitable hydrophobic or water insoluble polymers for controlled-release include, for example, methacrylic acid esters, ethyl cellulose, cellulose acetate, polyvinyl alcohol-maleic anhydride copolymers, β-pinene polymers, glyceryl esters of wood resins, and combinations comprising one or more of the foregoing polymers.

The inclusion of an effective amount of a plasticizer in the coating composition may improve the physical properties of the film. For example, because ethyl cellulose has a relatively high glass transition temperature and does not form flexible films under normal coating conditions, it may be advantageous to add plasticizer to the ethyl cellulose before using the same as a coating material. Generally, the amount of plasticizer included in a coating solution is based on the concentration of the polymer, e.g., most often from about 1 wt % to about 50 wt % of the polymer. Concentrations of the plasticizer, however, can be determined by routine experimentation.

Examples of plasticizers for ethyl cellulose and other celluloses include plasticizers such as dibutyl sebacate, diethyl phthalate, triethyl citrate, tributyl citrate, triacetin, and combinations comprising one or more of the foregoing plasticizers, although it is possible that other water-insoluble plasticizers (such as acetylated monoglycerides, phthalate esters, castor oil, etc.) can be used.

Examples of plasticizers for acrylic polymers include citric acid esters such as triethyl citrate NF, tributyl citrate, dibutyl phthalate, 1,2-propylene glycol, polyethylene glycols, propylene glycol, diethyl phthalate, castor oil, triacetin, and combinations comprising one or more of the foregoing plasticizers, although it is possible that other plasticizers (such as acetylated monoglycerides, phthalate esters, castor oil, etc.) can be used.

In certain embodiments, it is preferred that the coating is a substantially continuous coat and substantially hole-free. By substantially continuous coating is meant a coating which retains a smooth and continuous appearance when magnified 1000 times under a scanning electron microscope and wherein no holes or breakage of the coating are evident.

Suitable methods can be used to apply the coating to the dosage form. Processes such as simple or complex coacervation, interfacial polymerization, liquid drying, thermal and ionic gelation, spray drying, spray chilling, fluidized bed coating, pan coating, or electrostatic deposition, may be used. A substantially continuous nature of the coating may be achieved, for example, by spray drying from a suspension or dispersion of the coating composition.

The coatings may be about 0.005 micrometers to about 25 micrometers thick, preferably about 0.05 micrometers to about 5 micrometers.

In one embodiment, a carisoprodol dosage form is an oral dosage form such as, for example, a tablet. Oral dosage forms comprise about 100 mg to about 1000 mg of carisoprodol, specifically about 200 mg to about 400 mg of carisoprodol, and more specifically about 250 mg to about 350 mg of carisoprodol. In one embodiment, the oral dosage form is an immediate-release oral dosage form.

In one embodiment, a phenyloin dosage form is an oral dosage form such as, for example, a tablet. Oral dosage forms comprise about 20 mg to about 500 mg of phenyloin, specifically about 50 mg to about 200 mg of phenyloin, and more specifically about 100 mg of phenyloin. In one embodiment, the oral dosage form is an immediate-release oral dosage form.

### EXAMPLES

### Example 1

### Study of Phenyloin Pharmacokinetics when Phenyloin is Administered Alone or is Co-Administered with Carisoprodol as a Single-Dose

The study was designed as a randomized, single-dose two-way crossover to compare the pharmacokinetic parameters of carisoprodol (350 mg SOMA® (carisoprodol) Tablets by MedPointe Pharmaceuticals, MedPointe Healthcare Inc.) and phenyloin (PROMPT® Phenyloin Sodium 100 mg Capsules by IVAX Pharmaceuticals, Inc.). Twelve healthy adults participated in this comparison study and all of the subjects completed the study.

Subjects received two separate drug administration treatments in assigned periods, one treatment per period, according to the randomization schedule. Treatment (A) was 3 PROMPT® Phenyloin Sodium 100 mg capsules with 240 mL of room temperature water after an overnight fast. Treatment (B) was 3 PROMPT® Phenyloin Sodium 100 mg capsules and 2 tablets of SOMA® (carisoprodol 350 mg tablets) after an overnight fast.

Dosing days were separated by a washout period of at least fourteen days. Blood samples were drawn prior to dosing (pre-dose) and at 0.5, 1, 1.5, 2, 2.5, 3, 3.5, 4, 4.5, 5, 6, 7, 8, 10, 12, 18, 24, and 48 hours post-dose. The samples were then analyzed for total phenyloin. At study hours 2, 4, 6, 12 and 24 an additional blood sample was collected for the determination of “free” phenyloin.

The following pharmacokinetic parameters may be determined from the plasma concentration data:

The area under the plasma concentration versus time curve [AUCₜ] may be calculated using the linear trapezoidal rule from the zero time point to the last measured concentration.

The area under the plasma concentration versus time curve from zero to infinity [AUC_{0-INF}] may be calculated by adding Cₜ/Kₑₗₘ to AUC where Cₜ is the last measured concentration and Kₑₗₘ is the elimination rate constant.

The maximum observed plasma concentration [Cₘₐₓ] may be obtained by inspection.

The time to maximum plasma concentration [Tₘₐₓ] may be obtained by inspection. If the same maximum plasma concentration occurs at more than one time point, the first may be chosen as Tₘₐₓ.

The terminal elimination rate constant [Kₑₗₘ] may be obtained from the slope of the line, fitted by linear least squares regression, through the terminal points of the ln(base e) of the concentration versus time plot for these points.

The half-life [T_{1/2}] may be calculated by the equation T_{1/2}=0.693/Kₑₗₘ.

The data for free phenyloin when co-administered with carisoprodol is shown in Tables 1 and 2:

TABLE 1
Ln-transformed single-dose pharmacokinetic parameters for
free phenytoin when co-administered with carisoprodol

| | Phenytoin | | | 90% Confidence |
|---|---|---|---|---|
| | alone, | Phenytoin + | | Interval |
| | Geometric | Carisoprodol, | | (Lower limit, |
| | Mean | Geometric Mean | % Ratio | upper limit) |
| Cₘₐₓ | 282.76 | 322.25 | 87.75 | (69.5, 110.79) |
| (ng/ml) | | | | |
| AUC₀₋ₜ | 5150.25 | 5559.62 | 92.64 | (75.34, 113.9) |
| (hr*ng/ml) | | | | |
| AUC_{0-INF} | 12927.88 | 100673.26 | 121.12 | (0, 0) |
| (hr*ng/ml) | | | | |

TABLE 2
Non-transformed single-dose pharmacokinetic parameters for
free phenytoin when co-administered with carisoprodol

| | Phenytoin | Phenytoin + | |
|---|---|---|---|
| | alone, | Carisoprodol, | |
| | Least | Least | |
| | Sq. Mean | Sq. Mean | % Ratio |
| Tₘₐₓ (hr) | 9.17 | 8.68 | 105.6 |
| kₑₗₘ | 0.0279 | 0.0361 | 77.18 |
| T_{1/2} (hr) | 35.82 | 22.94 | 156.12 |

Single-dose co-administration of phenyloin and carisoprodol resulted in a 14% increase in the Cₘₐₓ for free phenyloin and an 8% increase in the AUC₀₋ₜ for free phenyloin.

The data for total phenyloin when co-administered with carisoprodol is shown in Tables 3 and 4:

TABLE 3
Ln-transformed single-dose pharmacokinetic parameters for
total phenytoin when co-administered with carisoprodol

| | Phenytoin | Phenytoin + | | 90% Confidence |
|---|---|---|---|---|
| | alone, | Carisoprodol, | | Interval |
| | Geometric | Geometric | | (Lower limit, |
| | Mean | Mean | % Ratio | upper limit) |
| Cₘₐₓ | 3222.2 | 2801.38 | 115.0 | (106.1, 124.7) |
| (ng/ml) | | | | |
| AUC₀₋ₜ | 97342.7 | 91468.8 | 106.4 | (96.35, 117.55) |
| (hr*ng/ml) | | | | |
| AUC_{0-INF} | 133987.2 | 132006.5 | 101.5 | (89.71, 114.84) |
| (hr*ng/ml) | | | | |

TABLE 4
Non-transformed single-dose pharmacokinetic parameters for
total phenytoin when co-administered with carisoprodol

| | Phenytoin | Phenytoin + | |
|---|---|---|---|
| | alone, Least | Carisoprodol, | |
| | Sq. Mean | Least Sq. Mean | % Ratio |
| Tₘₐₓ (hr) | 7.437 | 7.429 | 100.11 |
| Kₑₗₘ | 0.03395 | 0.033833 | 100.35 |
| T_{1/2} (hr) | 23.91 | 27.08 | 88.29 |

Single-dose co-administration of phenyloin and carisoprodol resulted in a 13% decrease in the Cₘₐₓ for total phenyloin, a 6% decrease in the AUC₀₋ₜ for total phenyloin, and a 1.5% decrease in the AUC_{0-INF} for total phenyloin.

The conclusion from these data is that when phenyloin and carisoprodol are co-administered, the Cₘₐₓ and AUC₀₋ₜ for free phenyloin increases when compared to administration of phenyloin alone. The Cₘₐₓ and AUC₀₋ₜ for free phenyloin co-administered with carisoprodol is not bioequivalent to phenyloin administered alone. In addition, the Cₘₐₓ and AUC₀₋ₜ for total phenyloin decrease somewhat when phenyloin is co-administered with carisoprodol. Thus, when co-administering carisoprodol and phenyloin, the blood level of free phenyloin should be measured.

### Example 2

### Study of Phenyloin Pharmacokinetics when Carisoprodol is Added to a Steady-State Regimen of Phenyloin

A single-arm, two-way drug interaction study was conducted in which carisoprodol (350 mg SOMA®) was added to a steady state regimen of phenyloin (PROMPT® Phenyloin Sodium 100 mg Capsules). The effects of coadministration of carisoprodol at steady state conditions on the Cₘₐₓ and AUC of phenyloin are summarized in Table 5. Twenty-four healthy adults participated in this comparison study and twenty-one of the subjects completed the study.

On study Days 1 through 14, a single 300 mg (1×300 mg capsule) dose of phenyloin sodium was administered to all study subjects following an overnight fast of at least 10 hours.

On study Days 15 through 28, a single 300 mg (1×300 mg capsule) dose of phenyloin sodium and a single 350 mg (1×350 mg tablet) dose of carisoprodol were co-administered to all study subjects following an overnight fast of at least 10 hours. A single dose of carisoprodol was re-administered to all study subjects 3 more times a day (every 6 hours) for Days 15 through 28.

Phenyloin: On study Days 14 and 28, 18 blood samples were collected (6 mL each) from each subject by direct venipuncture using pre-labeled Vacutainers® containing K₂ EDTA as an anticoagulant. Blood samples were collected within one hour prior to dosing and after dose administration at study hours 0.5, 1, 1.5, 2, 2.5, 3, 3.5, 4, 4.5, 5, 6, 7, 8, 10, 12, 18, and 24.

Approximately 216 mL of blood was collected from each subject for pharmacokinetic samples of phenyloin over the course of the study. The actual time at which each blood sample was collected was recorded by clinical staff.

Carisoprodol: On study Day 28, 12 blood samples were collected (4 mL each) from each subject by direct venipuncture using pre-labeled Vacutainers® containing K₃ EDTA as an anticoagulant. Blood samples were collected within one hour prior to dosing and after dose administration at study hours 0.5, 1, 1.5, 2, 2.5, 3, 3.5, 4, 4.5, 5, and 6.

Approximately 48 mL of blood was collected from each subject for pharmacokinetic samples of carisoprodol and meprobamate over the course of the study. The actual time at which each blood sample was collected was recorded by clinical staff.

TABLE 5
Ln-transformed pharmacokinetic parameters for free steady-state
phenytoin when co-administered with carisoprodol

| | Phenytoin | | | |
|---|---|---|---|---|
| | alone, | Phenytoin + | | 90% Confidence |
| | Day 14 | Carisoprodol, | | Interval |
| | Geometric | Day 28 | | (Lower limit, |
| | Mean | Geometric Mean | % Ratio | upper limit) |
| Cₘₐₓ | 929.76 | 1037.37 | 111.57 | (85.46, 145.68) |
| (ng/ml) | | | | |
| AUC₀₋ₜ | 14733.48 | 17416.69 | 118.21 | (90.88, 153.76) |
| (hr*ng/ml) | | | | |
| AUC_{0-INF} | 32701.49 | 45571.41 | 139.36 | (89.57, 216.8) |
| (hr*ng/ml | | | | |

TABLE 6
Non-transformed pharmacokinetic parameters for free steady-state
phenytoin when co-administered with carisoprodol

| | Phenytoin | Phenytoin + | |
|---|---|---|---|
| | alone, | Carisoprodol, | |
| | Day 14 | Day 28 | |
| | Least Sq. Mean | Least Sq. Mean | % Ratio |
| Tₘₐₓ (hr) | 5.04 | 5.05 | 100.14 |
| kₑₗₘ | 0.0312 | 0.0267 | 85.58 |
| T_{1/2} (hr) | 35.43 | 40.89 | 115.40 |

TABLE 7
Ln-transformed pharmacokinetic parameters for total steady-state
phenytoin when co-administered with carisoprodol

| | Phenytoin | | | |
|---|---|---|---|---|
| | alone, | Phenytoin + | | 90% Confidence |
| | Day 14 | Carisoprodol, | | Interval |
| | Geometric | Day 28 | | (Lower limit, |
| | Mean | Geometric Mean | % Ratio | upper limit) |
| Cₘₐₓ | 13604.17 | 15556.59 | 114.35 | (93.3, 140.15) |
| (ng/ml) | | | | |
| AUC₀₋ₜ | 242582.26 | 285541.72 | 117.71 | (93.17, 148.72) |
| (hr*ng/ml) | | | | |
| AUC_{0-INF} | 662587.00 | 843759.68 | 127.34 | (85.52, 189.62) |
| (hr*ng/ml | | | | |

TABLE 8
Non-transformed pharmacokinetic parameters for total steady-state
phenytoin when co-administered with carisoprodol

| | Phenytoin | Phenytoin + | |
|---|---|---|---|
| | alone, | Carisoprodol, | |
| | Day 14 | day 28 | |
| | Least Sq. Mean | Least Sq. Mean | % Ratio |
| Tₘₐₓ (hr) | 3.55 | 4.17 | 117.45 |
| kₑₗₘ | 0.0233 | 0.0215 | 92.47 |
| T_{1/2} (hr) | 39.42 | 42.65 | 108.20 |

The study results demonstrate both total phenyloin and free phenyloin AUC_{0-τ}, and Cₘₐₓ concentrations are increased (total phenyloin 18 and 14% and free phenyloin 18% and 12%) when steady-state phenyloin is administered with multiple-dose carisoprodol as compared to the total phenyloin and free phenyloin concentrations achieved when phenyloin is administered without carisoprodol under fasting conditions. The increases in phenyloin concentrations (total and free) reported in this study are clinically significant since phenyloin is a drug with a narrow therapeutic window. In one embodiment, it is recommended that steady-state free phenyloin concentrations are measured when carisoprodol is administered with steady-state phenyloin. In another embodiment, it is recommended that total and free phenyloin concentrations be monitored when carisoprodol is administered with steady-state phenyloin. Phenyloin dose adjustments may be necessary to avoid increased toxicity that can be associated with phenyloin concentrations above the therapeutic range (e.g., 10-20 μg/mL (total) and 1-2 μg/mL (free)).

The terms “a” and “an” do not denote a limitation of quantity, but rather denote the presence of at least one of the referenced item.

The term “or” means “and/or”.

The terms “comprising”, “having”, “including”, and “containing” are to be construed as open-ended terms (i.e., meaning “including, but not limited to”).

The endpoints of all ranges directed to the same component or property are inclusive and independently combinable.

Unless defined otherwise, technical and scientific terms used herein have the same meaning as is commonly understood by one of skill in the art to which this invention belongs.

Embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

We claim:

1. A method of increasing the serum level of free and/or total phenyloin in a patient, comprising
co-administering phenyloin and carisoprodol, wherein the phenyloin levels are at steady-state,
measuring the patient's free and total phenyloin levels 7-10 days after administering carisoprodol, and
adjusting the amount of carisoprodol administered or not adjusting the amount of carisoprodol administered so that the patient's free and/or total patient phenyloin level are about 1 to about 2 ug/mL for free phenyloin and about 10 to about 20 ug/mL for total phenyloin.

2. The method of claim 1, wherein the side effects include delirium, psychosis, encephalopathy, or irreversible cerebellar dysfunction.

3. A method of reevaluating dosing when administering carisoprodol to a patient in need of a skeletal muscle relaxant, comprising
co-administering carisoprodol and phenyloin to the patient, wherein the phenyloin levels are at steady-state;
determining whether the patient's free phenyloin level and total phenyloin level are within a safe range, wherein a safe range is about 1 to about 2 ug/mL for free phenyloin and about 10 to about 20 ug/mL for total phenyloin; and
increasing or decreasing the dose or frequency of carisoprodol administered to the patient if the free phenyloin levels, total phenyloin levels, or both, are outside the safe range.

4. The method of claim 3, further comprising stopping the carisoprodol administration and measuring the patient's free phenyloin level, total phenyloin level, or both.

5. A method of increasing the serum level of free and/or total phenyloin in a patient, comprising
co-administering phenyloin and carisoprodol, wherein the phenyloin levels are at steady-state,
measuring the patient's free and total phenyloin levels 7-10 days after administering carisoprodol, and
adjusting the amount of phenyloin administered or not adjusting the amount of phenyloin administered so that the patient's free and/or total patient phenyloin level are about 1 to about 2 ug/mL for free phenyloin and about 10 to about 20 ug/mL for total phenyloin.

6. A method of using phenyloin, comprising
co-administering phenyloin and carisoprodol, wherein the phenyloin levels are at steady-state,
measuring the patient's free and total phenyloin levels 7-10 days after administering carisoprodol, and
decreasing the amount of phenyloin administered so that the patient's free and/or total patient phenyloin level are about 1 to about 2 ug/mL for free phenyloin and about 10 to about 20 ug/mL for total phenyloin.

7. A method of preventing overdosing a patient with phenyloin, comprising:
administering carisoprodol daily for a period of at least about 14 days to the patient who is also being administered a dosage strength of phenyloin daily and
decreasing by about 85 to 89% the dosage strength of phenyloin being administered to the patient, and
optionally measuring the patient's blood plasma levels to determine if the patient's total phenyloin blood level is between about 10 to about 20 ug/mL.

8. The method of claim 7, wherein the dosage strength of phenyloin being administered to the patient is about 300 mg daily.

9. A method of preventing overdosing a patient with phenyloin, comprising:
administering carisoprodol daily for a period of at least about 14 days to the patient that is also being administered about 300 mg of phenyloin daily,
decreasing the phenyloin to between about 250 mg and about 270 mg, and
optionally measuring the patient's blood plasma levels to determine if the patient's total phenyloin blood level is between about 10 to about 20 ug/mL.

10. A method of decreasing the amount of phenyloin necessary to treat a patient in need of phenyloin, comprising,
administering carisoprodol for a period of at least about 14 days and
administering about 85% to about 89% of the amount of phenyloin administered to the patient.

11. The method of claim 10, wherein the amount of phenyloin administered to the patient is about 300 mg.

12. The method of claim 10, wherein the amount of carisoprodol administered to the patient is about 250 mg to about 350 mg.

13. A method of treating a patient in need of a skeletal muscle relaxant, comprising:
co-administering to the patient in need thereof carisoprodol and phenyloin, wherein the phenyloin is at steady-state, and
monitoring the level of free phenyloin in a patient serum sample during co-administration.

14. The method of claim 13, wherein monitoring improves the safety of treating the patient.

15. The method of claim 13, further comprising adjusting the dosage strength of phenyloin in response to the monitoring, adjusting the dosage frequency of phenyloin in response to the monitoring, observing the patient for signs of toxicity, or a combination thereof.

16. A method of preventing overdosing a patient with phenyloin, comprising:
administering a dose of carisoprodol to a patient having a steady-state blood level of phenyloin,
monitoring the level of free phenyloin in a patient serum sample during co-administration with carisoprodol, and
optionally adjusting the dose of phenyloin to maintain a safe level of phenyloin, wherein a safe level is about 1 to about 2 ug/mL for free phenyloin and about 10 to about 20 ug/mL for total phenyloin.

17. A method of determining a risk of overdosing a patient with phenyloin, comprising:
administering steady-state phenyloin to the patient,
determining that carisoprodol is administered to the patient, and
determining that the patient is at risk for an overdose of phenyloin if it is determined that the patient is administered carisoprodol with steady-state phenyloin.

18. The method of claim 17, further comprising adjusting the dosage strength of phenyloin in response to the monitoring, adjusting the dosage frequency of phenyloin in response to the monitoring, observing the patient for signs of toxicity, or a combination thereof.

19. A method of treating a patient in need of a skeletal muscle relaxant comprising:
co-administering steady-state phenyloin with carisoprodol,
measuring blood plasma concentrations of phenyloin from the patient 7 to 10 days after co-administering carisoprodol,
determining if free phenyloin, total phenyloin, or both increased,
making a record of the level of increase in phenyloin, and
reducing or stopping the dose of carisoprodol if the level of phenyloin increases such that the person is at risk of, or is illustrating side effects.

20. The method of claim 19, wherein the side effects include delirium, psychosis, encephalopathy, or irreversible cerebellar dysfunction.

21. A method of treating a patient in need of a skeletal muscle relaxant, comprising:
administering to the patient in need thereof a composition comprising carisoprodol, and
providing to the patient and/or a medical care worker published material providing information that when co-administering carisoprodol with steady-state phenyloin, the level of free phenyloin in a patient serum sample should be monitored during co-administration.

22. The method of claim 21, wherein the information provides that the level of free phenyloin in the patient serum is measured once every 7 to 10 days.

23. The method of claim 21, wherein the information provides that the level of free phenyloin in the patient serum is measured within 7 to 10 days.

24. The method of claim 21, further comprising determining if the patient is co-administered phenyloin with the carisoprodol.

25. The method of claim 24, further comprising monitoring the free phenyloin concentration in a serum sample from the patient when the patient is co-administered steady-state phenyloin with the carisoprodol.

26. The method of claim 21, wherein the published material includes information that co-administration of carisoprodol with steady-state phenyloin resulted in a 12% increase in the Cₘₐₓ for free phenyloin and an 18% increase in the AUC₀₋ₜ for free phenyloin during co-administration.

27. The method of claim 21, wherein the published material includes information that for co-administration of carisoprodol with steady-state phenyloin in a study of 21 patients,
the geometric mean of Cₘₐₓ for free phenyloin when phenyloin was administered alone was 929.76 ng/ml, and the geometric mean of Cₘₐₓ for free phenyloin when phenyloin was co-administered with carisoprodol was 1037.37 ng/ml during co-administration; and
the geometric mean of AUC₀₋ₜ for free phenyloin when phenyloin was administered alone was 14733.48 hr*ng/ml, and the geometric mean of AUC₀₋ₜ for free phenyloin when phenyloin was co-administered with carisoprodol was 17416.69 hr*ng/ml during co-administration.

28. The method of claim 27, further comprising providing information that co-administration of carisoprodol with steady-state phenyloin resulted in a 14% increase in the Cₘₐₓ for total phenyloin and an 18% increase in the AUC₀₋ₜ for total phenyloin during co-administration.

29. A method of preventing overdosing a patient with phenyloin, comprising:
co-administering to the patient steady-state phenyloin and a dose of carisoprodol, and
informing the patient and/or a medical care worker that co-administration of carisoprodol with steady-state phenyloin or can result in an increase in free phenyloin blood levels, total phenyloin blood levels, or both during co-administration.

30. The method of claim 29, further comprising
monitoring the free phenyloin concentration in a serum sample from the patient.

31. The method of claim 29, wherein the free phenyloin in the serum sample from the patient is measured once every 7 to 10 days.

32. The method of claim 29, wherein informing includes providing information that co-administration of carisoprodol with steady-state phenyloin resulted in a 12% increase in the Cₘₐₓ for free phenyloin and an 18% increase in the AUC₀₋ₜ for free phenyloin during co-administration.

33. The method of claim 29, wherein informing includes providing information that for co-administration of carisoprodol with steady-state phenyloin in a study of 21 patients,
the geometric mean of Cₘₐₓ for free phenyloin when phenyloin was administered alone was 929.76 ng/ml, and the geometric mean of Cₘₐₓ for free phenyloin when phenyloin was co-administered with carisoprodol was 1037.37 ng/ml during co-administration; and
the geometric mean of AUC₀₋ₜ for free phenyloin when phenyloin was administered alone was 14733.48 hr*ng/ml, and the geometric mean of AUC₀₋ₜ for free phenyloin when phenyloin was co-administered with carisoprodol was 17416.69 hr*ng/ml during co-administration.

34. The method of claim 29, wherein informing further includes providing information that co-administration of carisoprodol with steady-state phenyloin resulted in a 14% increase in the Cₘₐₓ for total phenyloin and an 18% increase in the AUC₀₋ₜ for total phenyloin during co-administration.

35. A method of using carisoprodol comprising:
informing a user that co-administration of carisoprodol with steady-state phenyloin can result in an increase in free steady-state phenyloin blood levels.

36. The method of claim 35, further comprising informing the user that when co-administering carisoprodol with steady-state phenyloin, the level of free phenyloin, the level of total phenyloin, or both, in a patient serum should be monitored during co-administration.
